(19) United States
(12) Patent Application Publication
(10) Pub. No.: US 2023/0364644 A1
(43) Pub. Date: Nov. 16, 2023

(54) ULTRASOUND TRANSDUCERS

(71) Applicant: Stelect Pty. Ltd., Richmond (AU)

(72) Inventors: Anurupa SHAW, Richmond (AU); Elise SUTHERLAND, Richmond (AU)

(21) Appl. No.: 18/361,805

(22) Filed: Jul. 28, 2023

Related U.S. Application Data
(63) Continuation of application No. 17/918,862, filed on Oct. 13, 2022.
(60) Provisional application No. 63/009,413, filed on Apr. 13, 2020.

(51) Int. Cl. B06B 1/06

(52) U.S. Cl. CPC B06B 1/0625

(57) ABSTRACT

Piezoelectric devices having small dimensions and which can operate at high frequencies with high penetration depths for a given applied voltage are described. The devices may be well suited for integration into medical devices, such as intravascular ultrasound (IVUS) catheters, to provide high resolution ultrasound images.

### CROSS REFERENCE TO RELATED APPLICATIONS

This application is a continuation of U.S. patent application Ser. No. 17/918,862, filed on Apr. 13, 2021, titled “ULTRASOUND TRANSDUCERS,” which is a national phase application under 35 U.S.C. 371 of International Patent Application No. PCT/IB2021/000250, filed on Apr. 13, 2021, titled “ULTRASOUND TRANSDUCERS,” now International Publication No. WO 2021/209816, which claims priority to U.S. Provisional Patent Application No. 63/009,413, filed on Apr. 13, 2020, entitled “ULTRASOUND TRANSDUCERS,” each of which is incorporated by reference herein in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD

Described herein are ultrasound transducers, in particular, piezoelectric micromachined ultrasonic transducer (PMUT) transducers, which may be configured as sensors (e.g., receivers and/or emitters).

### BACKGROUND

Ultrasonic transducers are used in a broad range of applications including range-finding applications, wind speed detection, ultrasonic baths, fingerprint sensors and ultrasonic medical imaging. The design of transducers can vary greatly depending on its use. Those used for ultrasonic imaging applications, for example, have very different focusing, sensitivity and power requirements than those used in range-finding applications.

Micromachined ultrasound transducers (MUTs) generally operate using one of two different mechanisms: capacitive force (CMUT) or piezoelectric (PMUT) sensing-actuation. Although CMUTs and PMUTs are both based on the flexural motion of a thin membrane, they have some principal differences. In CMUTs, energy transduction is due to change in capacitance, whereas in PMUTs, energy transduction is based on piezoelectricity of a piezoelectric material. More recently, increasing attention has been given to PMUTs as a potential solution for integrated transducer arrays due to their reduced power consumption and improved acoustic coupling compared to CMUTs. Despite these advantages, there are difficulties in manufacturing high performance thin films for PMUTs. It is also difficult to provide PMUTs having high penetration depths for penetrating through tissues in medical applications, including at high frequencies.

What is needed are improved PMUT devices that can operate at high frequencies and with high penetration depths, including those that can be implemented in small medical devices such as high resolution ultrasound imaging catheters.

### SUMMARY

Described herein are piezoelectric micromachined ultrasonic transducer (PMUT) apparatuses (e.g., devices, systems, assemblies, etc., including sensors) and methods of operating and making them. Compared to CMUT devices and conventional PMUT devices, the PMUT devices described herein can be smaller and can operate at relatively high frequencies with relatively high penetration depths for a given applied voltage. Thus, the devices may be well suited for integration into medical devices, such as intravascular ultrasound (IVUS) catheters, to provide high resolution ultrasound images.

The PMUT devices can include a number of piezoelectric stacks, arranged as a cell, with each cell including a multilayer stack extending proud of a base layer over a cavity in a substrate. The multilayer stack may include a plurality of piezoelectric layers, each flanked by electrode layers, and at least one base layer to add rigidity to the membrane during vibration. The thicknesses and/or materials of the piezoelectric layer(s) and/or base layer can be chosen to achieve a desired performance of the PMUT device. In some examples, the piezoelectric layers each have a height ranging from 0.25 micrometers to 3 micrometers. In some examples, the base layer has a thickness of at least 500 nanometers. In some examples, the one or more piezoelectric layers includes a lead-free material, such as zinc oxide and/or aluminum nitride.

Thus, the multilayer stack may include two or more piezoelectric layers, which may increase the total displacement of the multilayer stack membrane. As described herein, doubling of the piezoelectric layers may increase total displacement with unit driving voltage compared to a single stack of the same thickness. In cases where the multilayer stack includes two or more piezoelectric layers, the two or more piezoelectric layers may be arranged to have an alternating polarity to provide a uniform electric field inside the stack and for ease of connection, as adjacent piezoelectric layers may be separated by a single electrode. Alternatively, in some examples the piezoelectric layers may be polarized in the same direction, and may be sandwiched between separate electrode layers.

According to some examples, the piezoelectric stacks are arranged in a series of concentric rings, which may be referred to herein as a ring PMUTs. This arrangement may be configured as a bullseye pattern. The ring array arrangement can provide higher vibrational amplitude for a given driving voltage compared to single simple round or rectangular piezoelectric cell. The ring array structure may also provide better focusing compared to cells having a round or rectangular piezoelectric shape. In some cases, the arrangement having rings of piezoelectric stacks including two or more piezoelectric layers in the stack may increase the vibrational amplitude, penetration depth and focusing of the PMUT. In some examples, the PMUT device can have a working frequency between about 70 MHz and 80 MHz and a penetration depth of at least 0.6 cm (e.g., greater than 0.6 cm). In some examples, the PMUT device can have a working frequency between about 35 MHz and 45 MHz and a penetration depth of at least 1 cm. In some examples, the PMUT device can have a working frequency between about 10 MHz and 20 MHz and a penetration depth of at least 4 cm.

For example, described herein are piezoelectric ultrasound transducer devices comprising: a plurality of concentric multilayered stacks, each concentric multilayered stack extending proud of the base layer and the concentric multilayered stacks and base layer extending over a cavity, wherein the concentric multilayered stacks are separated by a space (e.g., which may be open so that air or other fluid may pass in the space), further wherein each of the concentric multilayered stacks includes a plurality of piezoelectric layers, and wherein each piezoelectric layer is arranged between electrode layers. In some examples the plurality of concentric multilayered stacks are formed by a single, long multilayered stack that spirals around itself to form the concentric shape.

In some examples, the piezoelectric ultrasound transducer device includes: a plurality of ring-shaped, concentric multilayered stacks, each concentric multilayered stack extending proud from a base layer, wherein the plurality of concentric multilayered stacks and the base layer extend over a cavity. The plurality of concentric multilayered stacks may be arranged in a bullseye pattern, further wherein each of the concentric multilayered stacks includes a plurality of piezoelectric layers, and wherein each piezoelectric layer is arranged between electrode layers, and wherein the piezoelectric layers within each stack are arranged in alternating polarity (e.g., so that the polarity of piezoelectric layers reverses along the height of the stack).

For example, a piezoelectric ultrasound transducer device may include: one or more multilayered stacks arranged concentrically over a base layer and extending proud of the base layer, wherein the one or more multilayered stacks and base layer are arranged over a cavity, further wherein each of the one or more multilayered stacks includes a plurality of piezoelectric layers arranged between electrode layers.

A piezoelectric ultrasound transducer device may include: one or more multilayered stacks arranged concentrically over a base in a spiral or bullseye pattern and extending proud of the base to a height, wherein the one or more multilayered stacks and base layer are arranged over a cavity in a substrate, further wherein each of the one or more multilayered stacks includes a plurality of piezoelectric layers arranged between electrode layers, such that the piezoelectric layers within the one or more multilayered stacks alternate in polarity along a direction from the base to the height.

The concentric multilayered stacks may include multiple piezoelectric layers, such as between 2-10 (e.g., between 2-3, between 2-4, between 2-5, between 2-6, between 2-7, between 2-8, between 2-10, etc.) piezoelectric layers. As mentioned, the piezoelectric layers within each stack may be arranged in alternating polarity. The polarity of the piezoelectric layers may be parallel to the polarity of an electric field applied when voltage is applied between the electrode layers.

This device may include any appropriate substrate, such as silicon and/or a silicon nitride base layer. In some examples the substrate may be considered separate from the silicon nitride base layer. The silicon nitride base layer may have a thickness of at least 100 nanometers (e.g., at least 200 nm, at least 300 nm, at least 400 nm, at least 500 nm, etc.).

The plurality of piezoelectric layers may comprise one or more of: a zinc oxide (ZnO), an aluminum nitride (AlN), an aluminum scandium nitride (AlScN), a lead magnesium niobate-lead titanate (PMN-PT) based material and a polyvinylidene difluoride (PVDF) polymer. As mentioned, in some examples the piezoelectric material comprises a zinc oxide (ZnO) or an aluminum nitride (AlN).

In some examples the direction of polarization of each piezoelectric layer is arranged parallel to a height of the piezoelectric layers (e.g., perpendicular to the base layer layer). Thus, when an electrical field is applied to the electrode layers on either side of each piezoelectric layer, the direction of polarization of the applied field is typically parallel with the direction of polarization of each piezoelectric layer (which may be opposite to each other).

The piezoelectric layers may each have a height ranging from, e.g., about 0.1 μm to about 5 μm (e.g., from about 0.25 μm to 3 about μm, from about 0.3 μm to about 2 μm, from about 0.4 μm to about 1 μm, etc.). Any of these apparatuses may have a range of operational frequencies (e.g., working frequencies) that is in the ultrasound range, e.g., between about 1 MHz and about 130 MHz, e.g., between about 5 MHz and about 100 MHz, between about 10 MHz and about 120 MHz, between about 10 MHz and about 100 MHz, between about 10 MHz and about 90 MHz, etc. For example, in some examples the apparatus has a working frequency between about 70 MHz and 80 MHz and has a penetration depth of at least 0.6 cm. In some examples, the apparatus has a working frequency between 35 MHz and 45 MHz and has a penetration depth of at least 1 cm. In some examples, the apparatus has a working frequency between 10 MHz and 20 MHz and has a penetration depth of at least 4 cm.

In any of these apparatuses, the transducer may have a calculated displacement of at least 0.1 percent of a sum of the piezoelectric layer thicknesses. In general, increasing the number of piezoelectric layers may substantially increase the total displacement for the same applied voltage.

In general, any of these apparatuses may include electrodes coupled to the electrode layers of the stack(s). All of the concentrically-arranged stacks may be coupled to the same pair(s) of electrodes. For example, any of these apparatuses may include a first electrical lead in electrical communication with a first half of the electrode layers in each of the multilayered stacks and a second electrical lead (which may be ground) in electrical communication with a second half of the electrode layers in each of the multilayered stacks. The electrode layers of the first half of the electrode layers in each of the multilayered stacks may alternate with the electrode layers of the second half of the electrode layers in each of the multilayered stacks.

Also described herein are methods of operating any of the piezoelectric ultrasound transducer apparatuses described herein. For example, a method of operating a piezoelectric ultrasound transducer apparatus may include: applying a voltage between a plurality of electrode layers in a piezoelectric ultrasound transducer, wherein the piezoelectric ultrasound transducer comprises a plurality of concentrically-arranged multilayered stacks each on a base layer over a cavity, further wherein each of the multilayered stacks includes a plurality of piezoelectric layers, and wherein each piezoelectric layer is arranged between pairs of electrode layers of the plurality of electrode layers; and inducing, from the applied voltage, a displacement that is a proportional to the applied voltage, the piezoelectric coefficient of the material forming the piezoelectric layers, and the number of piezoelectric layers.

The plurality of piezoelectric layers may be arranged so that a polarity of each of the piezoelectric layers alternate, further wherein applying the voltage comprises applying the voltage in a direction of polarization that is parallel with a direction of the polarity of each of the piezoelectric layers. The displacement is induced in the direction of polarization.

Applying the voltage between a plurality of electrode layers in the piezoelectric ultrasound transducer may comprise apply the voltage between a plurality of electrode layers in ring-shaped, concentric, multilayered stacks of the piezoelectric ultrasound transducer, wherein the concentric, multilayered stacks are arranged as a bullseye.

For example, inducing the displacement may comprise inducing a displacement at a frequency of between about 70 MHz and 80 MHz, wherein a penetration depth of an ultrasound signal emitted by the displacement is at least 0.6 cm. Inducing the displacement may comprise inducing a displacement at a frequency of between about 35 MHz and 45 MHz, wherein a penetration depth of an ultrasound signal emitted by the displacement is at least 1 cm. Inducing the displacement may comprise inducing a displacement at a frequency of between about 10 MHz and 20 MHz, wherein a penetration depth of an ultrasound signal emitted by the displacement is at least 4 cm.

For example, a method of operating a piezoelectric ultrasound transducer device may include: applying a voltage between a plurality of electrode layers in a piezoelectric ultrasound transducer in a direction of polarization, wherein the piezoelectric ultrasound transducer comprises a plurality of concentrically-arranged multilayered stacks each on a base layer over a cavity, further wherein each of the multilayered stacks includes a plurality of piezoelectric layers arranged so that the polarity of each piezoelectric layer (e.g., where polarity is induced and enforced by applying an electric field) alternates. The polarity of each of the piezoelectric layers may be parallel to the direction of polarization, and wherein each piezoelectric layer is arranged between pairs of electrode layers of the plurality of electrode layers; and inducing, from the applied voltage, a displacement in the direction of polarization that is a proportional to the applied voltage, the piezoelectric coefficient of the material forming the piezoelectric layers, and the number of piezoelectric layers.

Also described herein are methods of making (e.g., methods of forming) any of the apparatuses described herein. For example, a method of forming a piezoelectric ultrasound transducer having a plurality of concentric multilayer stacks extending proud of a base layer, wherein the base layer and the concentric multilayer stacks extend over a cavity, may include: forming a silicon nitride base layer on the substrate; forming a first electrode layer on the silicon nitride base layer; forming a first piezoelectric layer on the first electrode layer; forming a second electrode layer on the first piezoelectric layer; and forming one or more additional pairs of piezoelectric layers and electrode layers on the second electrode layer, wherein the first electrode layer, the first piezoelectric layer, the second electrode layer and the one or more additional pairs of piezoelectric layers and electrode layers is patterned into the plurality of concentric multilayer stacks. The concentric multilayered stacks may form a spiral (e.g., having a stack that is arranged in a concentric stack) or a plurality of concentrically-arranged rings.

The method may include lithographically patterning the first electrode layer, the first piezoelectric layer, the second electrode layer and the one or more additional pairs of piezoelectric layers and electrode layers into the plurality of ring-shaped multilayer stacks.

Any of these methods may include forming the corresponding cavity in the substrate. The first and second piezoelectric layers may be formed such that the first and second piezoelectric layers are arranged in alternating polarity.

The plurality of ring-shaped multilayer stacks may be concentrically arranged on the base layer to form a bullseye pattern.

In some examples, forming the piezoelectric material includes depositing a zinc oxide (ZnO) layer or an aluminum nitride (AlN) layer. In general, each of the piezoelectric layers may have a height ranging from, e.g., 0.1 μm to 5 μm (e.g., 0.25 micrometers to 3 micrometers, etc.). The silicon nitride base layer may be formed, e.g., by depositing the silicon nitride base layer to a thickness of at least 500 nanometers.

These and other examples, features and advantages are described herein.

Any of the PMUT cells described herein may be arranged as an array of PMUT cells, such as a linear array or a two-dimensional array. For example, described herein are catheters devices that include an array of PMUTs that may be used for imaging and/or sensing (via the ultrasound transducer) signals from within a body.

### BRIEF DESCRIPTION OF THE DRAWINGS

Novel features of examples described herein are set forth with particularity in the appended claims. A better understanding of the features and advantages of the examples may be obtained by reference to the following detailed description that sets forth illustrative examples and the accompanying drawings.

FIG. 1A illustrates a section view of an example piezoelectric stack showing a single piezoelectric layer.

FIG. 1B illustrates a perspective view and close-up view (inset) of a PMUT cell having concentrically arranged ring-shaped piezoelectric stacks similar to those shown in FIG. 1A.

FIG. 2A illustrates a section view of an example piezoelectric stack having two piezoelectric layers.

FIG. 2B illustrates a perspective view and close-up view (inset) of a PMUT having concentrically arranged ring-shaped piezoelectric stack of FIG. 2A.

FIG. 3 illustrates a stack of piezoelectric elements arranged in alternating polarity.

FIG. 4A is a graph showing calculated total displacement of a PMUT membrane achieved with different piezoelectric layer thicknesses.

FIG. 4B is a graph showing calculated total displacement of a PMUT membrane achieved with different bases layer thicknesses.

FIG. 5A is a graph showing calculated variation of the principal mode frequency of a single PMUT cell with respect to the radius of the piezoelectric layer.

FIG. 5B is a graph showing calculated variation of total displacement of the principal mode frequency of a single PMUT cell with respect to the radius of the piezoelectric layer.

FIG. 5C is graph showing calculated variation of the total displacement of the eigenmodes of a single PMUT cell have a particular cavity size.

FIG. 6A illustrates a simulation model based on calculated resonant modes and displacement fields for a ring PMUT with a single zinc oxide piezoelectric layer.

FIG. 6B illustrates a simulation model based on calculated resonant modes and displacement fields for a ring PMUT with a single aluminum nitride piezoelectric layer.

FIG. 7A is a simulation model showing total displacement of a ring array PMUT with a working resonant frequency of 13.54 MHz.

FIG. 7B is a simulation model showing total displacement of a ring array PMUT with a working resonant frequency of 42.92 MHz.

FIG. 7C is a simulation model showing total displacement of a ring array PMUT with a working resonant frequency of 78.98 MHz.

FIG. 7D is a simulation model similar to that shown in FIGS. 7A-7C, showing a cutaway region illustrating the displacement in the z-axis.

FIG. 8 is a graph comparing simulation results for calculated total displacement for resonant modes of a ring array PMUT having two piezoelectric layers and a ring array PMUT having a single piezoelectric layer of the same thickness.

FIG. 9 illustrates an acoustic field generated by an ultrasound transducer.

FIG. 10 shows a flowchart indicating a method for forming a PMUT device according to some examples.

FIG. 11A shows another example of a PMUT apparatus having concentrically-arranged multilayered stacks configured as a continuous spiral.

FIGS. 11B-11F, showing examples of PMUT apparatuses having concentrically-arranged multilayered stacks as described herein; FIG. 11B shows an example in which the concentrically-arranged multilayered stacks are formed from a continuous rectangular (e.g., square) spiral. FIG. 11C shows an example in which the concentrically-arranged multilayered stacks are formed from a continuous pentagonal spiral. FIG. 11D shows an example in which the concentrically-arranged multilayered stacks are formed from a continuous hexagonal spiral. FIG. 11E shows an example in which the concentrically-arranged multilayered stacks are formed from a continuous octagonal spiral. FIG. 11F shows an example in which the concentrically-arranged multilayered stacks are formed from a continuous polygonal spiral.

FIG. 12A shows a linear array of PMUTs as described herein.

FIG. 12B shows a ring array of PMUTS as described herein, configured as a side-viewing ring array.

### DETAILED DESCRIPTION

Described herein are piezoelectric micromachined ultrasonic transducer (PMUT) apparatuses (also referred to herein as PMUT devices, including PMUT sensors) with improved performance compared to CMUT devices and conventional PMUT devices. Compared to CMUT and conventional PMUT devices, the PMUT devices described herein can operate at relatively high frequencies and high penetration depth for a given applied voltage. These features can make the PMUT devices well suited for providing high resolution images when implemented with small medical imaging devices. The PMUT devices described herein may as components of a variety of devices and systems, including catheters (e.g., imaging catheters), fingerprint sensing, pipe sensors, etc. Although dimensions in exemplary examples are included herein, it is to be understood that these dimensions are merely to illustrate and not to limit, these apparatuses (devices and system). In particular, the PMUT devices described herein may be scaled up and/or the dimensions may be modified, including modifying to alter the frequency response ranges.

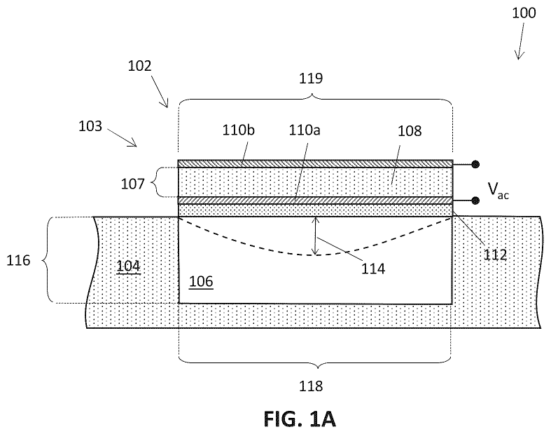

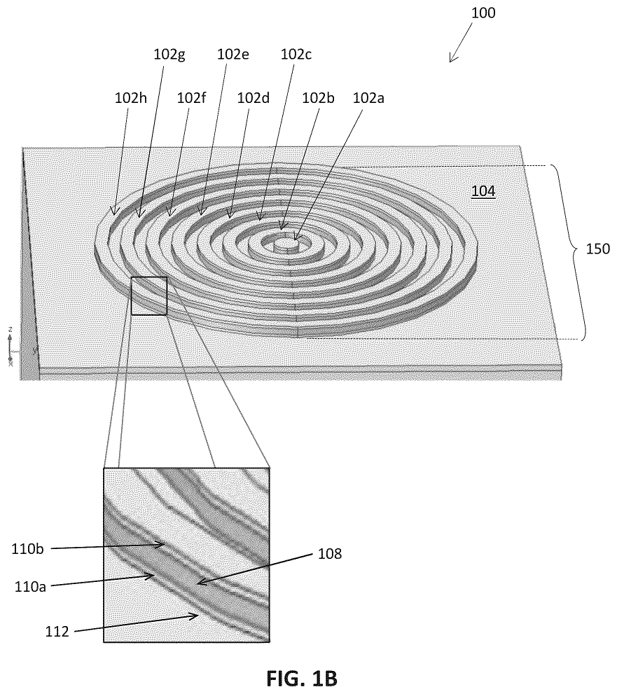

FIG. 1A shows a section view of a portion of an example piezoelectric stack 102 as part of a PMUT device cell 100. The piezoelectric stack 102 includes a multilayer stack 103, which acts as a vibrating membrane (also referred to as a diaphragm) formed over a corresponding cavity 106 in a substrate 104. The cavity within the substrate may be formed, e.g., using an etching process during the fabrication of the PMUT device. The multilayer stack membrane can include a first piezoelectric layer 108 made of a piezoelectric material. The piezoelectric layer can be situated between a first electrode layer 110a (e.g., bottom electrode layer) and a second electrode layer 110b (e.g., top electrode layer) that are operationally coupled to a power source (e.g., AC current source). A direction of polarization of the piezoelectric layer can be arranged parallel to a height 107 of the piezoelectric layer. The multilayer stack can also include a base layer 112 between one of the electrode layers (e.g., bottom electrode layer) and the substrate. When voltage is applied on the electrode layers, the piezoelectric layer converts the electrical energy to mechanical energy by vibrating at an excitation frequency. The excitation frequency can depend, in part, on the geometry of the piezoelectric cell. This vibration causes the suspended multilayer stack membrane to deflect 114 within the cavity of the substrate, thereby generating movement and force (e.g., movement in a direction that is perpendicular to the substrate/membrane of the device). In some examples the movement may be linear. In some examples the movement may not be linear (e.g., the base layer 112 can add rigidity to the membrane during the vibration, thereby affecting the degree of deflection of the membrane. As will be described in greater detail below, in general, any of these apparatuses may include multiple piezoelectric layers, although only one is shown in FIGS. 1A-1B.

According to some examples, the PMUT devices can include a number of ring-shaped piezoelectric stacks that are concentrically arranged (ring array), which can provide better focusing of the PMUT compared to simple round or rectangular PMUT structures. FIG. 1B shows an aerial view of the piezoelectric transducer 100 showing a concentrically arrangement of piezoelectric stacks 102a, 102b, 102c, 102d, 102e, 102f, 102g and 102h on the substrate 104. In this example, the piezoelectric stacks 102b-102h are ring-shaped and arranged concentrically about a center piezoelectric stack 102a having a circular shape. Each of the piezoelectric stacks 102a-102h can include the features of the piezoelectric stack 102 described above with respect to FIG. 1A. Each of the piezoelectric stacks 102a-102h includes a multilayer stack membrane, which includes the piezoelectric layer 108, electrode layers 110a and 110b, and base layer 112.

In general, these apparatuses may include one or more cavities. For example, in some examples, all of the multilayered stacks forming the cell of the apparatus may be arranged over a single cavity within the substrate. For example, the ring-shaped piezoelectric stacks 102b-102h can be arranged over a single cavity, and the circular-shaped piezoelectric stack 102a is arranged over the same cavity. The outer edge of the outer ring of the piezo electric stacks may positioned at the perimeter of the cavity.

Simulations show that a PMUT device having a ring array arrangement may provide improved performance in terms of vibration frequency compared to a PMUT device having a simple circular or rectangular piezoelectric cell of the same size (e.g., diameter), which can allow for better imaging resolution with the same penetration depth. For example, simulation results indicate that a ring array PMUT can provide higher vibrational amplitudes for 1 V driving voltage compared to a PMUT having a single circular cell. Examples of such simulations are described further below.

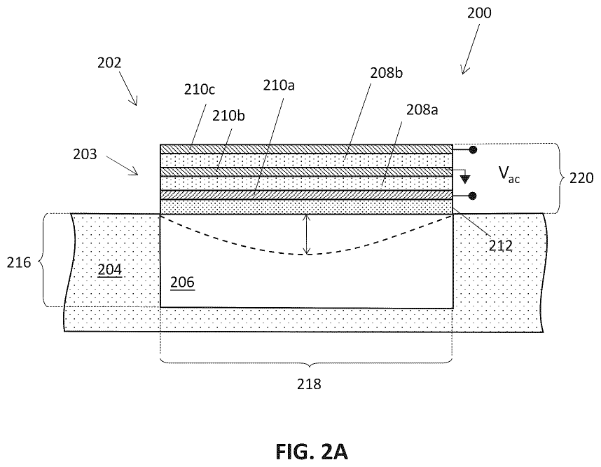

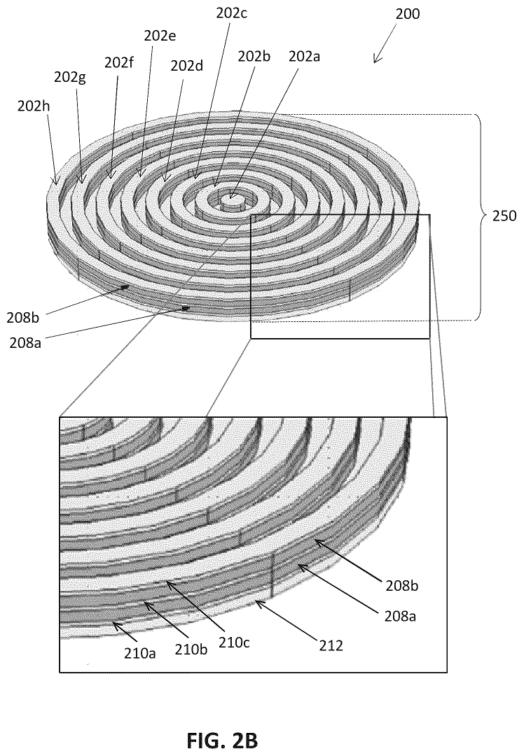

According to some examples, the PMUT devices include multiple piezoelectric layers. Multiple piezoelectric layers may be useful in ultrasound imaging applications since stacked piezoelectric layers can increase the vibrational amplitude and penetration depth of ultrasound in tissue compared to a PMUT having a single piezoelectric layer. For example, simulations have shown a PMUT having stacked piezoelectric layers in a ring array arrangement are shown to have a 150-times higher vibrational amplitude compared to a PMUT having a single piezoelectric layer in a ring array arrangement. FIG. 2A shows a section view of a portion of an example piezoelectric stack 202 as part of a PMUT device 200. The piezoelectric stack includes a multilayer stack membrane 203 formed over a corresponding cavity 206 in the substrate 204. In this example, the multilayer stack membrane 203 includes a first piezoelectric layer 208a (e.g., bottom piezoelectric layer) and a second piezoelectric layer 208b (e.g., top piezoelectric layer). The first piezoelectric layer 208a can be situated between a first electrode layer 210a (e.g., bottom electrode layer) and a second electrode layer 210b (e.g., middle electrode layer), and the second piezoelectric layer 208a can be situated between the second electrode layer 210b and a third electrode layer 210c (e.g., top electrode layer). A base layer 212 between one of the electrode layers (e.g., bottom electrode layer) and the substrate can provide rigidity to the membrane during deflection. FIG. 2B shows a broad perspective view of the PMUT 200 showing how multiple piezoelectric stacks 202a, 202b, 202c, 202d, 202e, 202f, 202g and 202h can be concentrically arranged, with each of the piezoelectric stacks 202a-202h including a piezoelectric layers 208a, 208b, electrode layers 210a, 210b, 210c, and base layer 212.

The example of FIGS. 2A and 2B show a PMUT device having two piezoelectric layers. However, the PMUT devices described herein can include any number of piezoelectric layers (e.g., 1, 2, 3, 4, 5, 6, or more layers). In some examples, the piezoelectric layers may have a maximum overall stack thickness 220 of the stacked membrane, depending on the particular application and size requirements. In theory, stacking of the piezoelectric layers can lead to a linear increase in amplitude of the vibration in theory, however, in practice fabrication errors may reduce the absolute amplitude. For example, the stack number may be increased from two to approximately 62. The optimal number of piezoelectric layers can be determined, e.g., by analyzing and quantifying the losses with the deposition of each stack. In some examples, the minimum number of piezoelectric layers is between 2-10, to achieve desired performance. In some examples, the PMUT device includes a stack having two to four piezoelectric layers.

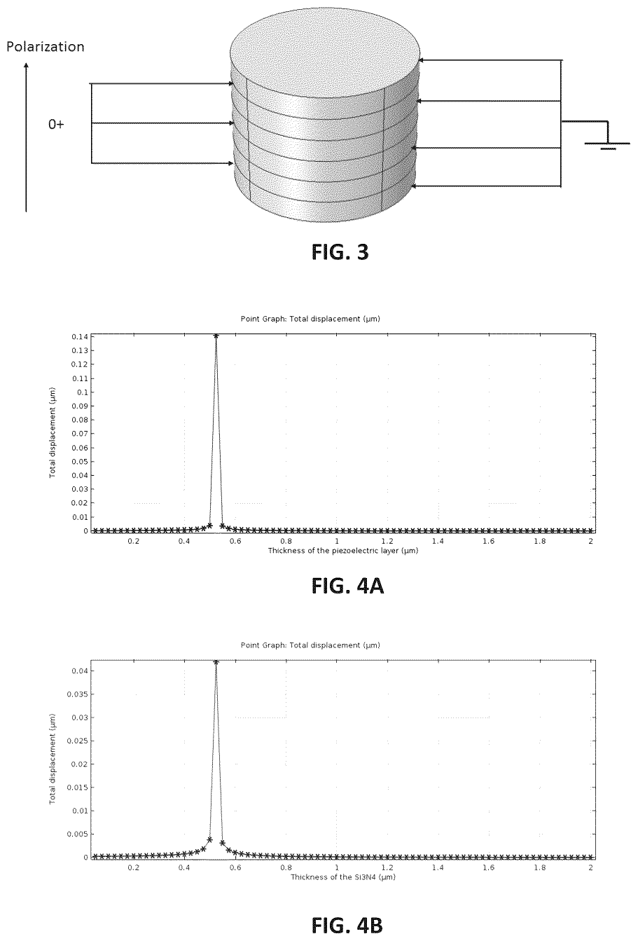

For PMUT devices having multiple piezoelectric layers, the piezoelectric layers may be arranged with alternating polarities. FIG. 3 shows an example of a stack of piezoelectric elements arranged in alternating polarity. When a voltage is applied parallel to the direction of polarization, a strain, or displacement, is induced in the direction of polarization. In FIG. 3, the every other electrode layer (arrows) is coupled to a first electrical lead (e.g., electrode), and the electrodes between those are connected to a second electrical lead (e.g., shown as ground in FIG. 3). Thus, the electrode layers in the multilayered stack shown to alternate, with every other electrode layer (moving from the base layer up the height of the stack) are in electrical communication with each other (e.g., connected to a common lead or electrode) and the remaining electrodes are also in electrical communication with each other (e.g., connected to a second common lead or electrode, in this case ground). The movement of a piezoelectric element equals the amount of voltage applied multiplied by the piezoelectric coefficient, D₃₃, which relates to the material's efficiency in transferring electrical energy to mechanical energy. Because the piezoelectric elements are connected mechanically in series, the total movement of a stacked piezoelectric actuator is the product of a single element's movement times the number of elements in the stack (ΔL=n*D₃₃*V, where ΔL is the change of length in meters (m), n=number of piezo layers, V=operating voltage, and D₃₃=longitudinal piezo electric coefficient (m/V)). The total displacement of a stacked actuator may be between 0.1 and 0.15 percent of the stack height. By stacking multiple piezoelectric layers, a higher displacement for a given voltage can be obtained compared to a single piezoelectric layer. The alternating polarity allows the maintaining of a uniform electric field inside the stack and also allows ease of connection.

The materials of the various components of the PMUT devices may vary depending, in part, on performance requirements and other requirements related to the particular application. In some examples, the piezoelectric material of the piezoelectric layer includes one or more of a zinc oxide (ZnO), an aluminum nitride (AlN), an aluminum scandium nitride (AlScN), a lead magnesium niobate-lead titanate (PMN-PT) based material, and a polyvinylidene difluoride (PVDF) polymer. In some medical device applications, for instance, a lead-free device may be required and may not utilize a PMN-PT piezoelectric material. The thickness (also referred to as “height”) (e.g., 107) of the piezoelectric layer(s) may vary depending, in part, on overall thickness requirements of the membrane (e.g., maximum thickness of the membrane) and the number of piezoelectric layers. In some examples where the device has a single piezoelectric layer (e.g., FIGS. 1A and 1B), the height of the piezoelectric layer may range from about 0.25 micrometers (μm) to about 3 μm (e.g., 0.25-3 μm, 0.5-2 μm, 0.5-1.5 μm, 0.75-1.25 μm, or 0.25-2 μm). The height of each piezoelectric layer in device that includes multiple piezoelectric layers may be less than the height of a piezoelectric layer having a single piezoelectric layer to avoid exceeding a maximum overall thickness of the membrane stack. In some examples where the device has multiple piezoelectric layers (e.g., FIGS. 2A and 2B), the height of each of the piezoelectric layers may range from about 0.1 (μm) to about 5 μm (e.g., about 0.1-4 μm, about 0.2-3 μm, about 0.25-2 μm, about 0.75-1.5 μm, about 0.5-2 μm, etc.). In some examples, the total height of the one or more piezoelectric layers (e.g., combined height if more than one piezoelectric layer) ranges from about 0.20 μm to about 5 μm (e.g., about 0.25-3 μm, about 0.5-2 μm, about 0.5-1.5 μm, about 0.75-1.25 μm, about 0.25-2 μm, etc.). As mentioned, these dimensions are only for illustration of particular examples; these dimensions may change based on the scale of the device and its intended frequency range.

In some examples, the base layer includes a piezo ceramic material. In some cases, the base layer includes silicon oxide (SiO₂) and/or silicon nitride (Si₃N₄). In some applications, a silicon nitride layer may be preferable as it may provide better responsiveness compared to silicon oxide for a given thickness.

The thickness of the base layer can depend, in part, on the thickness of the multilayer stack membrane. The base layer should be thick enough to provide sufficient rigidity to prevent the multilayer stack membrane from flexing too much and increasing the fragility of the device. However, the base layer should be thin enough to allow the multilayer stack membrane to sufficiently vibrate for piezoelectric functionality. For devices having a single piezoelectric layer (e.g. FIGS. 1A and 1B), the base layer thickness may range from about 200 nanometers (nm) to about 600 nm (e.g., about 200-600 nm, about 200-400 nm, about 300-400 nm, about 300-500 nm, etc.). For devices having two or more piezoelectric layers (e.g. FIGS. 2A and 2B), the base layer thickness may range from about 400 nm to about 700 nm (e.g., about 400-700 nm, about 400-600 nm, about 500-600 nm, about 500-700 nm, etc.). For devices having more than two piezoelectric layers, the base layer thickness may range, e.g., from about 500 nm to about 1000 nm (e.g., about 500-1000 nm, about 700-1000 nm, about 600-1000 nm, etc.). In some examples, for devices having two or more piezoelectric layers, the base layer may be at least 500 nm. These dimensions are for illustration only. As mentioned, the devices described herein may be scaled to larger or smaller dimensions based on the desired frequency characteristics and device use.

In some cases, the thickness of the piezoelectric layer(s) and the base layer are based on an eigenmode frequency of the device. FIGS. 4A and 4B are graphs showing results from 2D simulations based on a frequency mode of 76.75 MHz for a PMUT device. FIG. 4A shows a calculated total displacement of the membrane achieved for different thicknesses of the piezoelectric layer. These results indicate that a total piezoelectric layer(s) thickness between about 0.4 μm and 0.6 μm may provide optimal membrane displacement for a vibration frequency mode of 76.75 MHz. FIG. 4B shows a calculated total displacement of the membrane achieved for different thicknesses of the base layer. These results indicate that a base layer thickness between about 0.4 μm and 0.6 μm may provide optimal membrane displacement for a vibration frequency mode of 76.75 MHz.

The material of the electrode layers may vary depending on performance requirements and, in some cases, fabrication costs. In some examples, the electrode layers are made of or include aluminum, gold and/or platinum. In some examples, the electrode layers each include sublayers of different metals. For example, in some cases, each electrode layer includes a platinum layer and a titanium layer (e.g., 200:20 nm Pt/Ti), a platinum layer and a chromium layer (e.g., 200:20 nm Pt/Cr), a gold layer and a titanium layer (e.g., 200:20 nm Au/Ti), or a gold layer and a chromium layer (e.g., 200:20 nm Au/Cr). In some implementations, each electrode layer is made of different materials, which may provide good contrast in imaging applications. For instance, in some examples, a first electrode layer (e.g., bottom electrode layer) may be made of platinum and a second electrode layer (e.g., top electrode layer) may be made of gold. The thickness of the electrode layers may vary depending, in part, on the material(s) of the electrode layers. The electrode material should be thick enough to provide good adhesion and prevent acoustic losses, yet thin enough to avoid contributing too much to the overall thickness of the membrane. In some examples, each electrode layer can have a thickness ranging from about 100 nm to about 400 nm (e.g., 100-400 nm, 150-300 nm, 100-300 nm, or 200-400 nm).

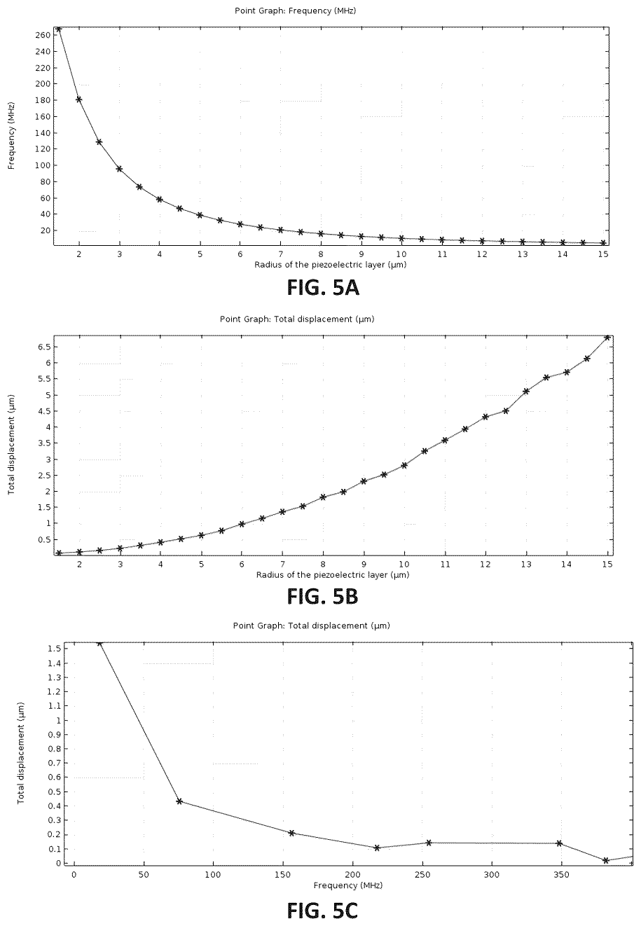

The dimensions of the various components of the PMUT devices may vary depending, in part, on performance requirements. FIGS. 5A-5C show results from a 2D simulation of a single PMUT cell to determine the effect of piezoelectric layer radius on vibration frequency and total displacement of a PMUT device. This information can be used estimate performance of a PMUT having a ring array arrangement. FIG. 5A is a plot showing the calculated variation of the principal mode frequency of a single PMUT cell with respect to the radius of the piezoelectric layer. FIG. 5B is a plot showing the calculated variation of total displacement of the principal mode frequency of a single PMUT cell with respect to the radius of the piezoelectric layer. The results indicate that the smaller the radius of the diaphragm of the device, the higher the principal mode frequency and the lower the vibration amplitude. FIG. 5C is a plot showing the calculated variation of the total displacement of the eigenmodes of a single PMUT cell have a particular cavity size (cavity radius of 15 μm with 40 μm by 40 μm piezoelectric cell dimensions) at different vibration frequencies. Note that even though the PMUT of FIG. 5C (having a cavity radius of 15 μm and 40 μm by 40 μm cell dimensions) is calculated to have a principal mode frequency of 14.4 MHz, the PMUT can be excited at a higher frequency mode (e.g., about 76.67 MHz) with a lower vibration amplitude. In some applications, a ring array PMUT device having piezoelectric stacks with the following dimensions are found to provide performance well suited for catheter sensing applications: a cavity height (e.g., FIG. 1A, 116) and piezoelectric layer radius/width (e.g., FIG. 1A, 119) ranging from about 50 μm to about 300 μm (e.g., 50-300 μm, 100-200 μm, or 75-150 μm), a cavity radius/width (e.g., FIG. 1A, 118) ranging from about 5 μm to about 20 μm (e.g., 5-20 μm, 10-20 μm, or 15-30 μm).

The number of piezoelectric stack rings and the ring pitch (distance between the rings) may also be selected based on simulations (e.g., 2D and/or 3D simulations) of a single PMUT cell. The example PMUT devices of FIGS. 1A-1B and 2A-2B include eight concentrically arranged piezoelectric stacks, however, the PMUT devices may include any number of concentrically arranged piezoelectric stacks (e.g., 1, 2, 3, 4, 5, 6, 7, 8, 9, 10, 11 or more). In some examples, the ring pitch ranges from about 0.25 μm to about 3 μm (e.g., 0.25-3 μm, 1-2 μm, 0.5-1.5 μm, or 1-2 μm). In one implementation, a ring pitch of 1 μm is maintained to allow eight stacks for a 30 μm PMUT cell. This can improve the tunability by enforcing the excitation frequency of the PMUT cell.

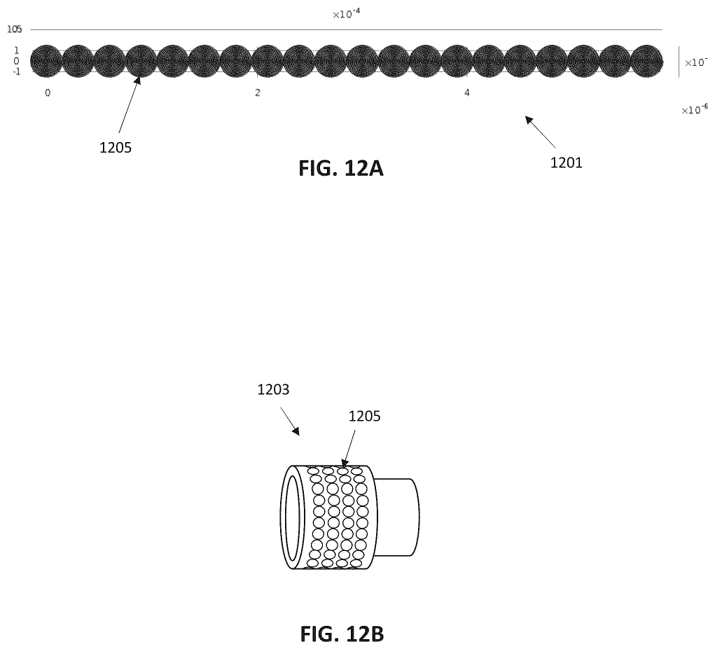

The overall dimensions of the PMUT device may be small so that one or more of the PMUT devices may be integrated within a small medical device, such as an ultrasound imaging catheter. For example, in some examples, as shown in FIG. 12A an array of PMUTs may be arranged on a device. In FIG. 12A, a linear array 1201 of PMUTS 1205 as described herein are shown. In this example an array of PMUTs as described herein form a line. In some examples the PMUT cells 1205 may be arranged on a device (e.g., as part of a catheter, for example) to form a side-facing array. For example, a line arrangement of a PMUT cells may be placed around a catheter with each line array (e.g., having an angular aperture of, for example, 5.625°) forming a side-viewing ring array. Other array configurations are possible, including side-viewing linear arrays, forward-viewing ring arrays, forward-viewing linear arrays, etc.). FIG. 12B illustrates an arrangement of PMUTs 1205 configured as a side-viewing ring array 1203. In another example, a ring array having a diameter from 5 μm to 35 μm, for a minimum of 64 elements, may be positions on a 3Fr catheter. Larger catheters may use larger ring arrays.

Any of the sensors (PMUT devices) described herein may generally have very small diameters. For example, each PMUT sensor may has a diameter that is 50 μm or less (e.g., 45 μm or less, 40 μm or less, 35 μm or less, 30 μm or less, 25 μm or less, 20 μm or less or less, etc.).

Another performance parameter of the PMUT device is penetration depth, which corresponds to the minimum scan depth at which electronic noise is visible, despite optimization of available controls (usually at the deepest transmit focal setting and maximum gain), and electronic noise stays at a fixed depth even when the PMUT is moved laterally. Penetration can primarily be determined by the center frequency of the transducer: the higher the frequency, the shallower the penetration because the absorption of the ultrasound wave traveling through tissue increases with frequency. A useful first approximation for estimating a depth of penetration (dp) for a given frequency is dp=60/f cm−MHz, where f is in MHz. The absorption coefficient (acoustic power loss per unit depth) is a function of frequency and varies from tissue to tissue (values for soft tissues range from 0.6 to 1.0 dB/cm−MHz). A more general term describing acoustic loss is the attenuation coefficient, which includes additional losses due to scattering and diffusion and hence is always greater than the absorption coefficient. The attenuation coefficient is highly patient and acoustic path dependent, hence it is difficult to simulate accurately. In order to have a simulation model that predicts it accurately, values can be extracted from experimental data and added to the models to obtain a robust model. The following are example PMUT devices.

### Example 1: Single Piezoelectric Layer PMUT Device

Simulations were performed based on a PMUT device having a ring array configuration with piezoelectric stacks having a single piezoelectric layer (FIGS. 1A and 1B) according to the specification in Table 1 below.

TABLE 1

| Number of piezoelectric layers | 1 | |
|---|---|---|
| Piezoelectric layer height | 1 | μm |
| Piezoelectric layer radius | 15 | μm |
| Base layer material | Si₃N₄ | |
| Base layer thickness | 300 | nm |
| Cell width | 40 μm × 40 μm | |
| Cavity radius | 15 | μm |
| Cavity height | 125 | μm |
| Electrode layers material | Al | |
| Electrode layer thickness (each) | 200 | nm |
| Ring pitch | 1 | μm |

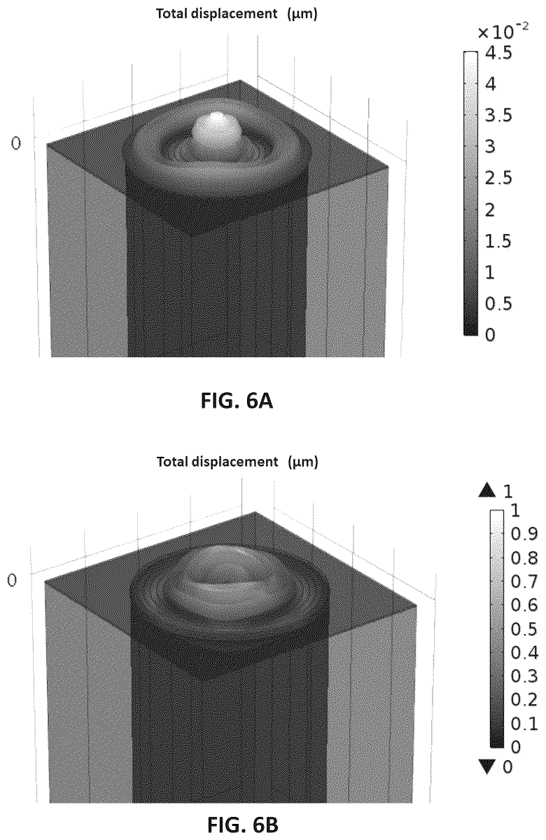

FIG. 6A shows results from a 3D simulation model based on calculated resonant modes and displacement fields for the PMUT with a single zinc oxide (ZnO) piezoelectric layer. A working frequency of 63.63 MHz and a total displacement of 50 nanometers (nm) is calculated using the ring PMUT with a ZnO piezoelectric layer, which is approximately a ten times greater in amplitude compared to a circular PMUT with a ZnO piezoelectric layer. FIG. 6B shows results from a 3D simulation model based on calculated resonant modes and displacement fields for a ring array PMUT with a single aluminum nitride (AlN) piezoelectric layer. A working frequency of 63.59 MHz and a total displacement of 1 micrometer (μm) is calculated using the ring PMUT with an AlN piezoelectric layer, which is approximately a 71 times greater in amplitude compared to a circular PMUT with an AlN piezoelectric layer.

### Example 2: Double Piezoelectric Layer PMUT Device

Simulations were performed based on a PMUT device having a ring array configuration with piezoelectric stacks having two piezoelectric layers (FIGS. 2A and 2B) according to the specification in Table 2 below.

TABLE 2

| Number of piezoelectric layers | 2 | |
|---|---|---|
| Piezoelectric layer height (each) | 0.5 | μm |
| Piezoelectric layer radius | 15 | μm |
| Piezoelectric layer material | ZnO | |
| Base layer material | Si₃N₄ | |
| Base layer thickness | 500 | nm |
| Cell width | 40 μm × 40 μm | |
| Cavity radius | 15 | μm |
| Cavity height | 125 | μm |
| Electrode layers material | Al | |
| Electrode layer thickness (each) | 200 | nm |
| Overall stack thickness | 1600 | nm |
| Ring pitch | 1 | μm |

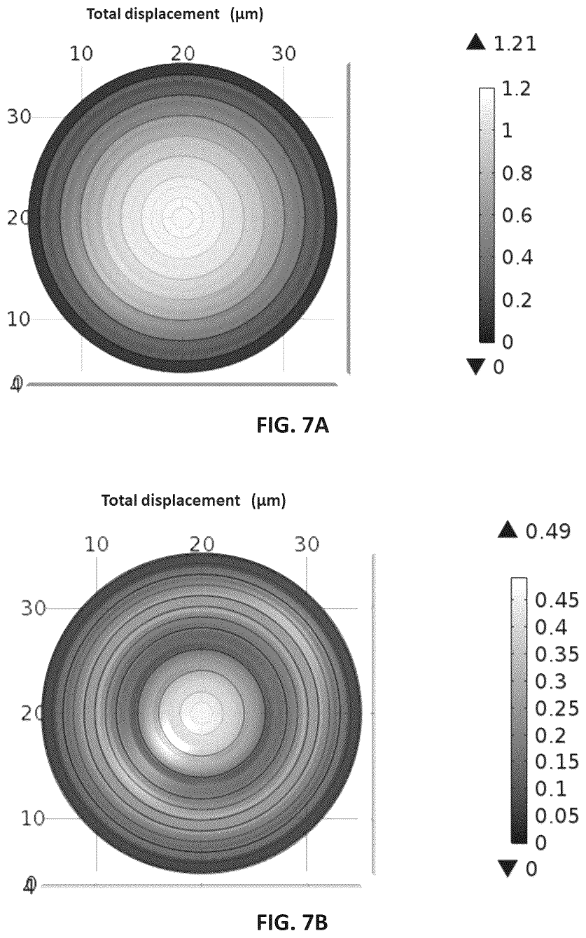

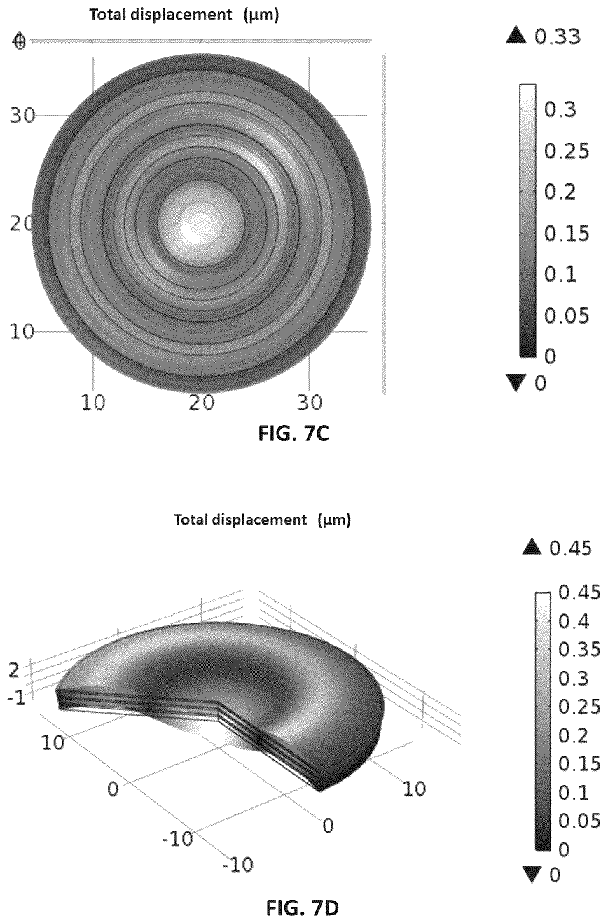

FIGS. 7A-7D illustrate simulation results showing total displacement (μm) of the ring array PMUT for three working resonant frequencies: 13.54 MHz (FIG. 7A), 42.92 MHz (FIG. 7B) and 78.98 MHz (FIGS. 7C and 7D). The calculated penetration depth of the PMUT device for three working resonant frequencies are summarized in Table 3 below.

TABLE 3

| | Calculated penetration |
|---|---|
| Frequency (MHz) | depth (60/frequency) |
| 13.54 | 4.43 cm-MHz |
| 42.92 | 1.39 cm-MHz |
| 78.98 | 0.75 cm-MHz |

The results indicate that the PMUT device can have a working frequency between about 10 MHz and 20 MHz and a penetration depth of at least 4 cm; a working frequency between about 70 MHz and 80 MHz and a penetration depth of at least 0.6 cm (e.g., greater than 0.6 cm); and/or a working frequency between about 35 MHz and 45 MHz and a penetration depth of at least 1 cm. Even the highest working frequency of the PMUT (around 70-80 MHz) provides a high penetration depth for a small device (e.g., for a 3 French catheter).

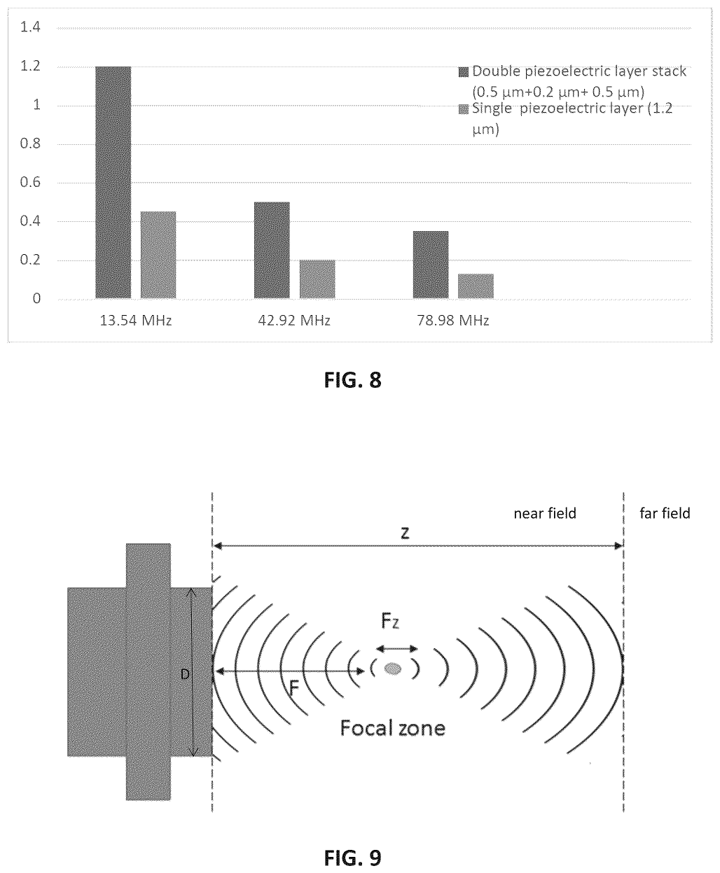

FIG. 8 is a graph comparing simulation results for calculated total displacement (μm) for resonant modes of a ring array PMUT having two ZnO piezoelectric layers and a ring array PMUT having a single ZnO piezoelectric layer of the same thickness. The simulation results indicate that the ring array PMUT having two ZnO piezoelectric layers provides a gain in frequency that provides higher resolution and the doubling of the piezoelectric stack provides an increase in total displacement with unit driving voltage compared to a single piezoelectric stack of the same thickness.

In some applications, the PMUT devices are implemented in ultrasound imaging catheters. The high penetration depth and small size of the piezoelectric transducers described herein can make the transducers well suited for integrating into/onto the small diameter catheters. In general, the focal length of a transducer is the distance from the face of the transducer to the point in the sound field where the signal with the maximum amplitude is located. In an unfocused transducer, this occurs at a distance from the face of the transducer which is approximately equivalent to the transducer's near field length. FIG. 9 shows a schematic representation of an acoustic field generated by an ultrasound transducer. Because the last signal maximum occurs at a distance equivalent to the near field, a transducer cannot be acoustically focused at a distance greater than its near field. The near field distance N (shown as “Z” in FIG. 9) is calculated as:

N=D²f/4c

where D is the transducer diameter, f is the frequency, and c is the speed of sound in the medium (in blood, 1540 m/s). The focal distance F is the distance between the transducer and the focal point that is the target zone. Individual PMUT cells (e.g., simple round or rectangular shaped cells) may not be focused, hence their focal length can be considered to be the length of their near field. PMUT cells assembled in an array (e.g., as a ring array of stacks), such as the ring arrays described herein, can produce the focusing effect. The calculated near field distance N for the PMUT having two piezoelectric layers (Example 2) at different resonant modes of interest are provided in Table 4 below.

TABLE 4

| | Near field distance | |
|---|---|---|
| Frequency (MHz) | (N) of a single cell | |
| 13.54 | 1.9782 | μm |
| 42.92 | 6.2708 | μm |
| 78.98 | 11.5397 | μm |

The calculated results indicate that the PMUT device can have a working frequency between about 10 MHz and 20 MHz and a near field distance of about 1 μm to about 3 μm; a working frequency between about 70 MHz and 80 MHz and a near field distance of about 5.5 μm to about 6.5 μm; and/or a working frequency between about 35 MHz and 45 MHz and a near field distance of about 10.5 μm to about 12.5 μm.

One or more of the ring array PMUT devices can be incorporated in and/or on the catheters. In some cases, the one or more transducers form a circular ring around the catheter. In some cases, one or more transducers are on the exterior walls of the imaging catheter, for example, at or near a distal end of the catheter. In such an arrangement, the transducer(s) may capture images along the side of the catheter (e.g., radially outward from a central axis of the catheter) to provide a side view along the catheter. Alternatively or additionally, the one or more transducers may be positioned at the distal tip of the imaging catheter. In such an arrangement, the transducer(s) may be configured as a sensor to capture images from the front (e.g., distal tip) of the catheter for a forward view from the catheter. In some cases, the transducers for use in medical imaging can be lead-free. Thus, for example, the piezoelectric material may be made non-lead-based materials, such as zinc oxide.

The PMUT devices described herein can include those having any number of shapes and arrangements, and are not limited to the examples of FIGS. 1A-1B and 2A-2B. In some examples, the piezoelectric cells may include concentrically arranged polygonal (e.g., square, triangular, rectangular, pentagonal or hexagonal), elliptical or oval shaped rings rather than circular-shaped rings. In some examples, one or more piezoelectric stacks may have spiral/helical shape that winds from the center of the transducer. Different shapes and configurations may provide certain directionalities to the transducers, which may be useful in certain applications. However, certain shapes may increase the complexity of the design and fabrication of the devices. Thus, simpler configurations may be desirable and may provide suitable performance for certain applications.

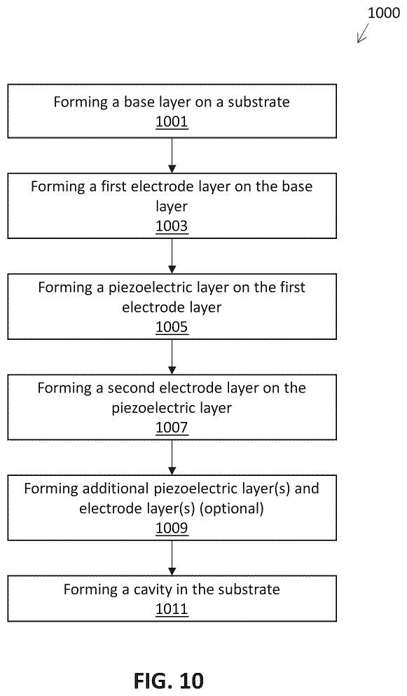

FIG. 10 shows a flowchart 1000 indicating a method of forming piezoelectric cells of a PMUT device according to some examples. A variety of different fabrication techniques may be use. In one example, to form the piezoelectric cells in accordance with a ring array structure, each of the processes 1001-1011 can be performed on the substrate (e.g., wafer) in accordance with the ring array pattern (e.g., FIG. 1B or 2B). At 1001, a base layer can be formed on the substrate. In some cases, the base layer is formed by a deposition process, such as plasma-enhanced chemical vapor deposition (PECVD) or low pressure chemical vapor deposition (LPCVD). In some cases, the substrate (e.g., silicon) is cleaned or etched prior to deposition. For example, a thermal oxide may be removed using an acidic solution (e.g., hydrofluoric acid). In some cases, a base layer is deposition on both sides—to be used as the mask for the substrate (e.g., silicon) anisotropic wet etching. At 1003, a first (e.g., bottom) electrode layer is formed on the base layer. In some examples, the base layer is deposited by ion-beam sputtering, patterned by photolithographic techniques, and wet etched (e.g., using H₃PO₄ solution). Alternatively or additionally, the multi-layer stack may be deposited on the substrate (e.g., all the layers) and the patterns may be etched for the individual electrodes and the piezoelectric layers one at a time.

At 1005, a piezoelectric layer is formed on the first electrode layer. In some examples, the piezoelectric layer is formed using a sputtering process, such as a magnetron sputtering process. In some cases, the piezoelectric layer is deposited to achieve a crystalline structure conducive with providing good piezoelectric properties. For example, a ZnO layer may exhibit a densely packed structure with columnar crystallites preferentially orientated along the (002) plane. In some cases, the piezoelectric layer is patterned (e.g., by wet etching using H₃PO₄ solution). At 1007, a second (e.g., top) electrode layer is formed on the piezoelectric layer. In some examples, the second electrode layer is deposited by ion-beam sputtering and photolithographically patterned by lift-off processing. At 1009, the method may optionally involve forming one or more additional piezoelectric layer and electrode layers to form a PMUT having multiple piezoelectric layers. This can involve repeating processes 1005 and 1007.

At 1011, a cavity is formed in the substrate. In some cases, forming the cavity involves a number of processes. In some cases, forming the cavity involves a back side film (e.g., Au/Cr) deposition, where the back side film is deposited on the back side of the wafer and patterned by back-to-front alignment photolithography techniques and a wet etching process, followed by inductively coupled plasma dry etching of based layer to form the mask for substrate (e.g., silicon) wet etching. In some cases, forming the cavity involves a back side mask etching process, where the wafer substrate is anisotropically etched using an etchant (e.g., KOH etchant at 70 C) to release the diaphragm. In some cases, forming the cavity involves a bulk machining process, where the bulk substrate material (e.g., silicon) is etched (e.g., wet etched) until the required cavity thickness is achieved. In some cases, forming the cavity involves a back side oxide etching, where oxide is removed by acidic solution (e.g., hydrofluoric acid solution) from the diaphragm. In some cases, fabrication of the PMUT device includes a wafer washing process, where the wafer is washed (e.g., with deionized water) after unloading from a fixture.

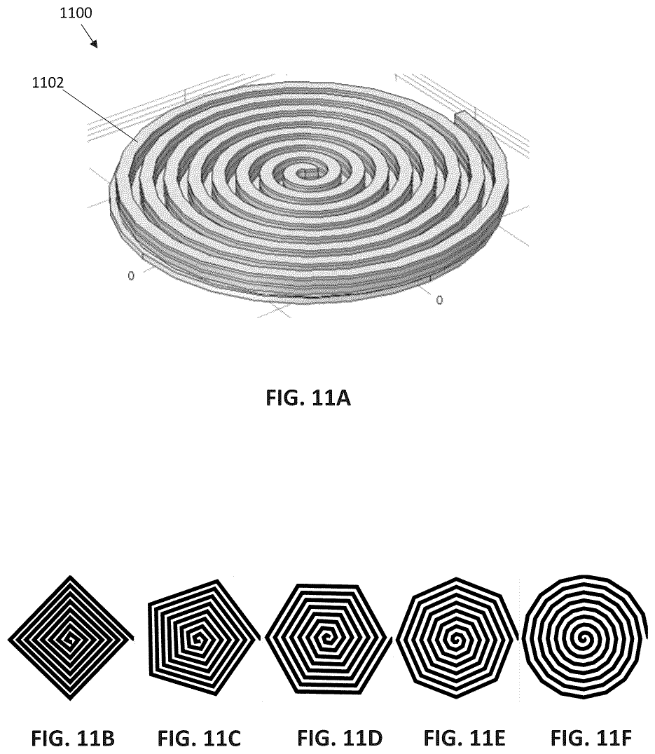

As mentioned above, the apparatuses described herein generally include a plurality of concentrically-arranged multilayered stacks. In some examples (e.g., shown in FIGS. 1B and 2B) the concentrically-arranged multilayered stacks are formed from a plurality of separate ring-shaped, concentric, multilayered stacks. Alternatively, in some examples the concentrically-arranged multilayered stacks may be formed from a continuous spiral, as shown in FIGS. 11A and 11B-11F. For example, in FIG. 11A, the cell is formed of a single spiral forming the concentrically-arranged multilayered stacks 1102. The spiral wraps outward with a constant pitch (center to center distance between 2 electrodes), constant electrode thickness and width. Thus, the spiral shown in FIG. 11A has eight loops that are concentrically arranged (and continuous with each other).

Similarly, FIGS. 11B-11F show other examples of ultrasound transducer apparatuses (e.g., PMUT devices) as described herein, in which the plurality of concentrically-arranged multilayered stacks on the substrate are formed from a polygonal spiral. FIGS. 11A-11F all show different polygonal shapes of the PMUT cell where the number of sides of the PMUT cell in 4 in FIG. 11B, 5 in FIG. 11C, 6 in FIG. 11D, 8 in FIGS. 11E, and 16 in FIG. 11F. Thus, a plurality of concentrically-arranged multilayered stacks may refer to two or more separate and concentrically arranged stacks, a single concentrically spiraling multilayered stack, multiple concentrically spirally multilayered stacks or a combination of a concentrically spirally stack and one or more separate encircled/encircling stacks.

In any of the apparatuses described herein the plurality of concentrically-arranged multilayered stacks may be separated by a gap (e.g., an air gap or space) between the concentrically arranged stacks. In FIGS. 11A-11F the gap is approximately the same width as the stack; in some examples the gap may be larger or smaller.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one example, the features and elements so described or shown can apply to other examples. It will also be appreciated by those of skill in the art that references to a structure or feature that is disposed “adjacent” another feature may have portions that overlap or underlie the adjacent feature.

Terminology used herein is for the purpose of describing particular examples only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

In general, any of the apparatuses and methods described herein should be understood to be inclusive, but all or a sub-set of the components and/or steps may alternatively be exclusive, and may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative examples are described above, any of a number of changes may be made to various examples without departing from the scope of the invention as described by the claims. For example, the order in which various described method steps are performed may often be changed in alternative examples, and in other alternative examples one or more method steps may be skipped altogether. Optional features of various device and system examples may be included in some examples and not in others. Therefore, the foregoing description is provided primarily for exemplary purposes and should not be interpreted to limit the scope of the invention as it is set forth in the claims.

The examples and illustrations included herein show, by way of illustration and not of limitation, specific examples in which the subject matter may be practiced. As mentioned, other examples may be utilized and derived there from, such that structural and logical substitutions and changes may be made without departing from the scope of this disclosure. Such examples of the inventive subject matter may be referred to herein individually or collectively by the term “invention” merely for convenience and without intending to voluntarily limit the scope of this application to any single invention or inventive concept, if more than one is, in fact, disclosed. Thus, although specific examples have been illustrated and described herein, any arrangement calculated to achieve the same purpose may be substituted for the specific examples shown. This disclosure is intended to cover any and all adaptations or variations of various examples. Combinations of the above examples, and other examples not specifically described herein, will be apparent to those of skill in the art upon reviewing the above description.

What is claimed is:

1. A piezoelectric ultrasound transducer device comprising:
a plurality of ring-shaped, concentric multilayered stacks extending proud of a base layer to a height, wherein the plurality of concentric multilayered stacks and base layer are arranged over a cavity, wherein the concentric multilayered stacks are arranged in a bullseye pattern, further wherein each of the multilayered stacks includes a plurality of piezoelectric layers, and wherein each piezoelectric layer is arranged between electrode layers, such that the piezoelectric layers within each concentric multilayered stack alternate in polarity along a direction of the height of each concentric multilayered stack.

2. The device of claim 1, wherein the concentric multilayered stacks each includes between two and eight piezoelectric layers.

3. The device of claim 1, wherein the substrate comprises silicon.

4. The device of claim 1, wherein the base layer comprises a silicon nitride base layer.

5. The device of claim 1, wherein the silicon nitride base layer has a thickness of at least 500 nanometers.

6. The device of claim 1, wherein the plurality of piezoelectric layers comprise one or more of: a zinc oxide (ZnO), an aluminum nitride (AlN), an aluminum scandium nitride (AlScN), a lead magnesium niobate-lead titanate (PMN-PT) based material and a polyvinylidene difluoride (PVDF) polymer.

7. The device of claim 1, wherein a direction of polarization of each piezoelectric layer is arranged parallel to a direction of each concentric multilayered stack perpendicular to the base layer.

8. The device of claim 1, wherein the piezoelectric layers each have a thickness ranging from 0.25 micrometers to 3 micrometers.

9. The device of claim 1, wherein the device has a working frequency between about 70 MHz and 80 MHz and has a penetration depth of at least 0.6 cm.

10. The device of claim 1, wherein the device has a working frequency between 35 MHz and 45 MHz and has a penetration depth of at least 1 cm.

11. The device of claim 1, wherein the device has a working frequency between 10 MHz and 20 MHz and has a penetration depth of at least 4 cm.

12. The device of claim 1, wherein the device has a calculated displacement of at least 0.1 percent of a height of the one or more piezoelectric layers.

13. The device of claim 1, further comprising a first electrical lead in electrical communication with a first subset of the electrode layers in each concentric multilayered stack and a second electrical lead in electrical communication with a second subset of the electrode layers in each concentric multilayered stack.

14. The device of claim 13, wherein in each concentric multilayered stack electrode layers of the first subset of the electrode layers alternate with electrode layers of the second subset of the electrode layers.

15. A method of operating a piezoelectric ultrasound transducer device, the method comprising:
applying a voltage between a plurality of electrode layers in a piezoelectric ultrasound transducer, wherein the piezoelectric ultrasound transducer comprises a plurality of concentric multilayered stacks, each multilayered stack extending proud of a base layer over a cavity, further wherein each of the concentric multilayered stacks includes a plurality of piezoelectric layers, and wherein each piezoelectric layer is arranged between two electrode layers of the plurality of electrode layers; and
inducing, from the applied voltage, a displacement that is proportional to the applied voltage, the piezoelectric coefficient of the material forming the piezoelectric layers, and the number of piezoelectric layers.

16. A piezoelectric ultrasound transducer device comprising:
one or more multilayered stacks arranged concentrically over a base in a spiral or bullseye pattern and extending proud of the base to a height, wherein the one or more multilayered stacks and base layer are arranged over a cavity in a substrate, further wherein each of the one or more multilayered stacks includes a plurality of piezoelectric layers arranged between electrode layers, such that the piezoelectric layers within the one or more multilayered stacks alternate in polarity along a direction from the base to the height.
